(19) United States
(12) Patent Application Publication
(10) Pub. No.: US 2024/0389920 A1
(43) Pub. Date: Nov. 28, 2024

(54) VISION TRANSFORMER SYSTEM AND METHOD CONFIGURED TO GENERATE A PATIENT DIAGNOSIS FROM AN ELECTROCARDIOGRAM

(71) Applicant: ICAHN SCHOOL OF MEDICINE AT MOUNT SINAI, New York, NY (US)

(72) Inventors: Akhil Vaid, New York, NY (US); Girish N. Nadkarni, New York, NY (US)

(73) Assignee: ICAHN SCHOOL OF MEDICINE AT MOUNT SINAI, New York, NY (US)

(21) Appl. No.: 18/672,348

(22) Filed: May 23, 2024

Related U.S. Application Data
(60) Provisional application No. 63/468,435, filed on May 23, 2023.

(51) Int. Cl. A61B 5/316; A61B 5/00; G16H 50/20

(52) U.S. Cl. CPC A61B 5/316; A61B 5/7267; G16H 50/20

(57) ABSTRACT

A vision transformer system and method generate a diagnosis from an electrocardiogram (ECG) of the patient. A patch generating module generates image patches of the ECG. A tokenization module generates numerical patch-based tokens corresponding to image patches. A transformer module generates a numerical classification token from the numerical patch-based tokens. A classification module generates and outputs a diagnosis message from the numerical classification token, wherein the diagnosis message is the patient diagnosis corresponding to the patient ECG and indicating a state of health of the heart of the patient. A masking module mask a preset portion of the plurality of patches, and the numerical classification token is generated from the plurality of numerical patch-based tokens, the unmasked patches, and the masked patches. The tokenization module receives ECG training data to be trained to generate the numerical classification token. The method implements the vision transformer system.

### CROSS-REFERENCE TO RELATED APPLICATION

This application claims priority to pending U.S. Provisional Patent Application No. 63/468,435, filed May 23, 2023, which is incorporated herein by reference in its entirety.

### GOVERNMENT SUPPORT CLAUSE

This invention was made with government support under DK107908 awarded by the National Institute of Health. The government has certain rights in the invention.

### FIELD OF THE DISCLOSURE

The present disclosure relates generally to performing a diagnosis of a patient using an electrocardiogram (ECG or EKG), and, more particularly, to a vision transformer system and method configured to generate a patient diagnosis from an electrocardiogram of the patient.

### BACKGROUND OF THE DISCLOSURE

An electrocardiogram (ECG or EKG) is a body surface level recording of electrical activity within the heart. Due to low cost, non-invasiveness, and wide applicability of ECGs to diagnose cardiac disease, the ECG is a ubiquitous investigation tool, and over 100 million ECGs are performed each year within the United States alone in various healthcare settings. However, the ECG is limited in scope since physicians cannot consistently identify patterns representative of disease, especially for conditions which do not have established diagnostic criteria, or in cases when such patterns may be too subtle or chaotic for human interpretation.

Machine learning such as deep learning has been applied to process ECG data for several diagnostic and prognostic use cases. The vast majority of use cases employ convolutional neural networks (CNNs). As with other types of neural networks, CNNs are high variance constructs, and require large amounts of data to prevent overfitting. CNNs must also be purpose built to accommodate the dimensionality of incoming data, and CNNs have been used for interpreting ECGs both as one-dimensional (1D) waveforms and two-dimensional (2D) images.

In this context, interpreting ECGs as 2D images presents an advantage due to widely available pre-trained models which often serve as starting points for modeling tasks on smaller datasets. This technique is described as transfer learning wherein a model that is trained on a larger, possibly unrelated dataset is fine-tuned on a smaller dataset that is relevant to a problem. Transfer learning is especially useful in healthcare since datasets are limited in size due to limited patient cohorts, rarity of outcomes of interest, and costs associated with generating useful labels. As a result, vision models first trained in a supervised manner on natural images often form the basis of models used in healthcare settings. Unfortunately, transfer learning with such natural images is not a universal solution, and it is known to produce suboptimal results when there exist substantial differences in the pre-training and fine-tuning datasets.

In the prior art, applying machine learning methods and models, such as convolutional neural networks (CNNs), to evaluating ECGs has been unreliable in providing accurate diagnoses of the heart of a patient, since certain pathological patterns of an ECG such as the S1Q3T3 occur in different parts of an ECG recording. Such patterns as the S1Q3T3 may represent right heart strain, also known as right ventricular (RV) strain, which is a medical finding of right ventricular dysfunction. Machine learning methods and models which consider only contiguous regions of the ECG may miss such pathological patterns entirely.

### SUMMARY OF THE DISCLOSURE

According to an implementation consistent with the present disclosure, a vision transformer system and method are configured to generate a patient diagnosis from an electrocardiogram of the patient.

In an implementation, a vision transformer system is configured to generate a patient diagnosis. The vision transformer system comprises a hardware-based processor, a memory, and a set of modules. The memory is configured to store instructions and configured to provide the instructions to the hardware-based processor. The set of modules is configured to implement the instructions provided to the hardware-based processor. The set of modules includes a patch generating modules, a tokenization module, a transformation module, and a classification module. The patch generating module is configured to generate a plurality of patches by partitioning an image of a patient electrocardiogram (ECG) having at least one patient ECG waveform into a plurality of sub-images, wherein each patch is a respective one of the plurality of sub-images and further wherein each patch has fewer pixels than the image of the patient ECG. The tokenization module is configured to generate, using a predetermined tokenization algorithm, a plurality of numerical patch-based tokens, wherein each of the numerical patch-based tokens is a numerical value representing a respective one of the plurality of patches. The transformer module is configured to generate, by processing the plurality of numerical patch-based tokens, a numerical classification token representing the patient ECG. The classification module is configured to generate and output, by processing the numerical classification token, a diagnostic message representing a patient diagnosis corresponding to the patient ECG and indicating a state of health of the heart of the patient.

The transformer module can include a first neural network that is trained using ECG training data including an image of at least one training ECG having at least one training ECG waveform. The first neural network can be trained by repeatedly evaluating the ECG training data until a respective first training generated diagnosis differs from an actual first training diagnosis within a first predetermined training threshold. The classification module can include a second neural network that is trained using the ECG training data. The second neural network can be trained by repeatedly evaluating the ECG training data until a respective second training generated diagnosis differs from an actual second training diagnosis within a second predetermined training threshold.

The classification module can comprise a multi-layer perceptron classification module including the second trained neural network. The transformer module can comprise a multi-head attention module, and a multi-layer perceptron module. The multi-head attention module can be configure to perform a predetermined attention transformation on the plurality of numerical patch-based tokens. The multi-layer perceptron module can include the first trained neural network configured to generate the numerical classification token from the transformed plurality of numerical patch-based tokens.

The vision transformer system can further comprise a masking module configured to generate, by masking a subset of the plurality of patches, a plurality of masked patches. The transformer module can be configured to generate the numerical classification token using the plurality of numerical patch-based tokens, a plurality of unmasked patches, and the plurality of masked patches. Each of the masked patches can include pixels having a predetermined color. The masking module can include an optimizer configured to perform stochastic optimization with a predetermined learning rate to define the subset of the plurality of patches. The tokenization module can include a generative pre-trained transformer configured to convert each of the plurality of patches to respective ones of the plurality of numerical patch-based tokens.

In another implementation, a pre-trained vision transformer system is configured to generate a patient diagnosis. The vision transformer system comprises a hardware-based processor, a memory, and a set of modules. The memory is configured to store instructions and configured to provide the instructions to the hardware-based processor. The set of modules is configured to implement the instructions provided to the hardware-based processor. The set of modules includes a patch generating module, a tokenization module, a pre-trained transformer module, and a pre-trained classification module. The patch generating module is configured to generate a plurality of patches by partitioning an image of a patient electrocardiogram (ECG) having at least one patient ECG waveform into a plurality of sub-images, wherein each patch is a respective one of the plurality of sub-images and further wherein each patch has fewer pixels than the image of the patient ECG. The tokenization module is configured to generate, using a predetermined tokenization algorithm, a plurality of numerical patch-based tokens, wherein each of the numerical patch-based tokens is a numerical value representing a respective one of the plurality of patches. The pre-trained transformer module is trained using ECG training data including an image of at least one training ECG with at least one training ECG waveform, and is configured to generate, by processing the plurality of numerical patch-based tokens, a numerical classification token representing the patient ECG. The pre-trained classification module is trained using the ECG training data and is configured to generate and output, by processing the numerical classification token, a diagnostic message representing a patient diagnosis corresponding to the patient ECG and indicating a state of health of the heart of the patient.

The pre-trained transformer module can include a first neural network that is trained using the ECG training data, and the first neural network can be trained by repeatedly evaluating the ECG training data until a respective first training generated diagnosis differs from an actual first training diagnosis within a first predetermined training threshold. The pre-trained classification module can include a second neural network that is trained using the ECG training data. The second neural network can be trained by repeatedly evaluating the ECG training data until a respective second training generated diagnosis differs from an actual second training diagnosis within a second predetermined training threshold.

The classification module can comprise a multi-layer perceptron classification module including the second trained neural network. The transformer module can comprise a multi-head attention module and a multi-layer perceptron module. The multi-head attention module can be configured to perform a predetermined attention transformation on the plurality of numerical patch-based tokens. The multi-layer perceptron module can include the first trained neural network configured to generate the numerical classification token from the transformed plurality of numerical patch-based tokens.

The pre-trained vision transformer system can further comprise a masking module configured to generate, by masking a subset of the plurality of patches, a plurality of masked patches. The transformer module can be configured to generate the numerical classification token using the plurality of numerical patch-based tokens, a plurality of unmasked patches, and the plurality of masked patches. Each of the masked patches can include pixels having a predetermined color. The masking module can include an optimizer configured to perform stochastic optimization with a predetermined learning rate to define the subset of the plurality of patches. The tokenization module can include a generative pre-trained transformer configured to convert each of the plurality of patches to respective ones of the plurality of numerical patch-based tokens.

In a further implementation, a computer-based method comprises receiving an electrocardiogram (ECG) of the patient, wherein the patient ECG includes a plurality of pixels representing an image having at least one patient ECG waveform. The computer-based method further comprises generating a plurality of patches of the patient ECG by partitioning the image of the patient ECG into a plurality of sub-images, wherein each patch is a respective one of the plurality of sub-images and further wherein each patch has fewer pixels than the image of the patient ECG. The computer-based method further comprises generating a plurality of numerical patch-based tokens from the plurality of patches using a predetermined tokenization algorithm, wherein each numerical patch-based token is a numerical value representing a respective one of the plurality of patches. The computer-based method further comprises generating a numerical classification token by processing the plurality of numerical patch-based tokens using a transformer module having a first trained neural network, wherein the numerical classification token represents the patient ECG. The computer-based method further comprises generating a diagnosis message from the numerical classification token processed by a classification module including a second trained neural network, wherein the diagnosis message represents a patient diagnosis corresponding to the patient ECG and indicating a state of health of the heart of the patient. The computer-based method further comprises outputting the diagnosis message. The computer-based method can further comprise providing a first neural network in the transformer module, providing a second neural network in the classification module, training the first neural network using ECG training data including an image of at least one training ECG having at least one training ECG waveform, and training the second neural network using the ECG training data. The training of the first neural network can include repeatedly evaluating the ECG training data until a respective first training generated diagnosis differs from an actual first training diagnosis within a first predetermined training threshold. The training of the second neural network can include repeatedly evaluating the ECG training data until a respective second training generated diagnosis differs from an actual second training diagnosis within a second predetermined training threshold.

Any combinations of the various embodiments, implementations, and examples disclosed herein can be used in a further implementation, consistent with the disclosure. These and other aspects and features can be appreciated from the following description of certain implementations presented herein in accordance with the disclosure and the accompanying drawings and claims.

### BRIEF DESCRIPTION OF THE DRAWINGS

FIG. 1 is a schematic of a vision transformer system, according to an implementation.

FIG. 2 is a schematic of a computing device used in the implementation of FIG. 1.

FIG. 3 is a schematic of a transformer module used in the implementation of FIG. 1.

FIG. 4 is a schematic of a neural network used in the implementation of FIG. 1.

FIG. 5 is a flow diagram of the processing of data by the components of the vision transformer system of FIG. 1.

FIG. 6 is an example of an ECG.

FIG. 7 is an example of the ECG partitioned into a plurality of patches.

FIG. 7A is an example of a patch representing a portion of an ECG waveform.

FIG. 8 is an example of a masking of a portion of the plurality of patches in FIG. 7.

FIG. 9 is an example of tokens generated from the plurality of patches in FIG. 7.

FIG. 10 is an example of masked patches in FIG. 7.

FIGS. 11A-11B are flowcharts of operation of the vision transformer system of FIG. 1.

For the purpose of illustrating the invention, there are depicted in drawings certain embodiments and implementations of the invention. However, the invention is not limited to the precise arrangements and instrumentalities of the embodiments and implementations depicted in the drawings.

It is noted that the drawings are illustrative and are not necessarily to scale.

### DETAILED DESCRIPTION OF CERTAIN EMBODIMENTS OF THE DISCLOSURE

Example embodiments and implementations consistent with the teachings included in the present disclosure are directed to a vision transformer system 100 and method 1100 configured to generate a patient diagnosis from an electrocardiogram of the patient.

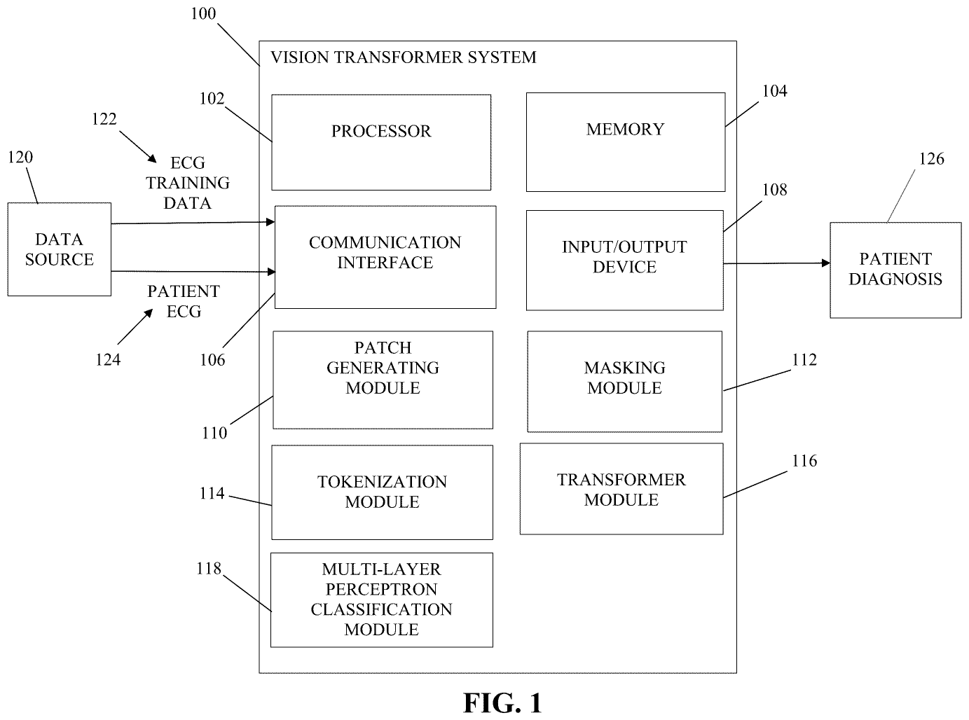

Transformer based neural networks utilize an attention mechanism to establish and define relationships between discrete units of input data known as tokens. A significant benefit that transformers allow for is unsupervised learning from large corpuses of unlabeled data to learn relationships between tokens, and then utilize this information for other downstream tasks. Due to the case with which unstructured text can be broken down into tokens, transformers have been tremendously successful in performing Natural Language Processing (NLP) tasks. In an implementation consistent with the invention, the vision transformer system 100 in FIG. 1 has extended the functionality of such NLP models into vision-based tasks.

The vision transformer system 100 of FIG. 1 implements a Bidirectional Encoder representation from Image Transformers (BeiT) approach to allow large unlabeled datasets to be leveraged for pre-training transformer neural networks. This approach consists of converting parts of an input image into discrete tokens or patches. Such tokens may be considered analogous to the words within a sentence and may be used to pre-train a transformer in much the same way as with a language model. Since transformers consider global dependencies between all features of provided inputs, such pre-training of the vision transformer system 100 may be especially advantageous for ECGs. As described below, the vision transformer system 100 is pre-trained on a large corpus of several million ECGs belonging to a diverse population. The vision transformer system 100 is applicable to use cases where little data may be available.

Referring to FIG. 1, in an implementation consistent with the invention, the vision transformer system 100 includes a hardware-based processor 102, a memory 104 configured to store instructions and configured to provide the instructions to the hardware-based processor 102, a communication interface 106, an input/output device 108, and a set of modules 110-118 configured to implement the instructions provided to the hardware-based processor 102. The set of modules 110-118 includes a patch generating module 110, a masking module 112, a tokenization module 114, a transformer module 116, and a multi-layer perceptron (MLP) classification module 118. In an implementation, the instructions are code written in the 3.8.x version of the Python programming language. In another implementation, the instructions are code written in any known programming language.

In one implementation, the vision transformer system 100 is operatively connected to a data source 120 through a network. For example, the network is the Internet. In another example, the network is an internal network or intranet of an organization. In a further example, the network is a heterogeneous or hybrid network including the Internet and the intranet. The data source 120 transmits, conveys, or otherwise provides ECG training data 122 and a patient ECG 124 to the vision transformer system 100. The patient ECG 124 includes a plurality of pixels representing an image having at least one patient ECG waveform. The ECG training data 122 includes a plurality of discrete ECG images as ECG recordings obtained from a plurality of subjects, with each ECG recording of the ECG training data 112 includes a plurality of pixels representing an image having at least one subject ECG waveform.

In one implementation, the data source 120 is in proximity to the vision transformer system 100. In another implementation, the data source 120 is remote from the vision transformer system 100. In a further implementation, the ECG training data 122 is obtained from a database of ECGs. For example, the ECG training data 122 includes a corpus of 8.5 million discrete ECG recordings obtained from 2.1 million patients. In one implementation, the ECG training data 122 are formatted as structured extensible markup language (XML) files including both raw waveforms as well as metadata associated with patient identifiers, time, place, indication, and characteristics such as diagnoses of the patients associated with each of the ECG training data 122. In another implementation, the ECG training data 122 are formatted in any known data format.

In one implementation, the patient ECG 124 is obtained in real time from a patient using an electrocardiogram device. Such a real time patent ECG 124 is temporarily or permanently stored in the data source 120, and is transmitted, conveyed, or otherwise provided to the communication interface 106 of the vision transformer system 100.

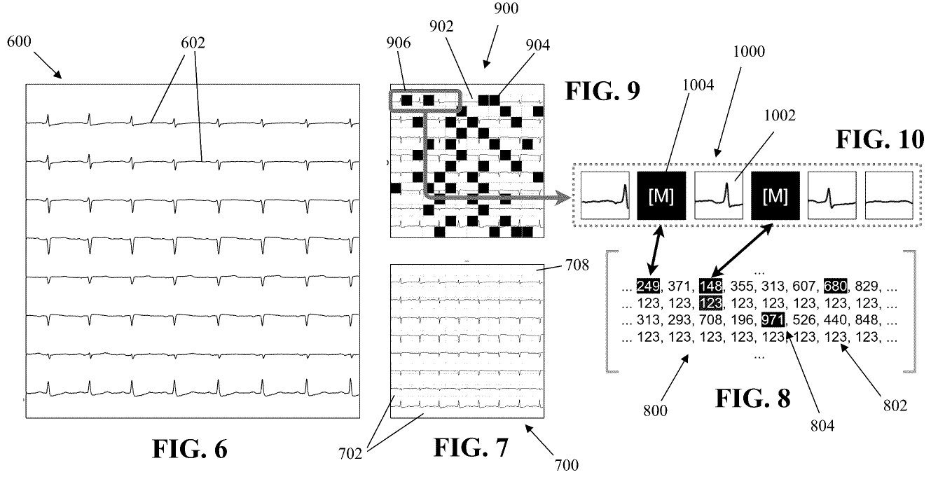

At least the transformer module 116 of the vision transformer system 100 is trained by the ECG training data 122. Optionally, the MLP classification module 118 is also trained by the ECG training data 122. For example, as shown in FIG. 6, the ECG training data 122 and the patient ECG 124 include an ECG 600 having at least one ECG waveform 602.

Once the transformer module 116 and optionally the MLP classification module 118 are trained by the ECG training data 122, the vision transformer system 100 is configured to process the patient ECG 124 and to generate and output a patient diagnosis 126 corresponding to the patient ECG 124. Accordingly, the vision transformer system 100 is configured to diagnose the health of a patient corresponding to the patient ECG 124. For example, based on the patient ECG 124, the diagnosis 126 generated and output by the vision transformer system 100 indicates that the corresponding patient has a healthy heart. In another example, based on the patient ECG 124, the diagnosis 126 generated and output by the vision transformer system 100 indicates that the corresponding patient has a healthy heart, or the corresponding patient has an unhealthy heart, such as hypertrophic cardiomyopathy, low left ventricular ejection fraction, or ST elevation myocardial infarction. In one implementation, the vision transformer system 100 generates a numerical value representing the diagnosis 126, such as a real number within a predetermined range of, for example, zero to one, or a percentage-based real number within a predetermined range of, for example, zero to one hundred. For example, a diagnosis 126 having a numerical value of over 60% indicates a healthy heart, while a diagnosis 126 having a numerical value of less than or equal to 60% indicates an unhealthy heart, such that 60% is a default cut-off value. In another implementation, a system administrator, using the input/output device 108, sets or changes the default cut-off value to a different percentage value.

In one implementation, the patient diagnosis 126 is an alert, a notification, or a message output from the input/output device 108. For example, the input/output device 108 includes a display or monitor configured to visually display the patient diagnosis 126 to a doctor, an ECG technician, or a patient. The patient diagnosis 126 is a text message or an image representing the state of the heart of the patient, such as the patient corresponding to the patient ECG 124, as having a healthy heart, or the corresponding patient has an unhealthy heart such as hypertrophic cardiomyopathy, low left ventricular ejection fraction, or ST elevation myocardial infarction.

In another example, the input/output device 108 includes an audio speaker configured to output an audible sound, corresponding to the patient diagnosis 126, to a doctor, an ECG technician, or a patient. In a further example, the input/output device 108 include both a display and an audio speaker, and the patient diagnosis 126 include a video or animation with audio conveying that the patient, corresponding to the patient ECG 124, has a healthy heart, or the corresponding patient has an unhealthy heart such as hypertrophic cardiomyopathy, low left ventricular ejection fraction, or ST elevation myocardial infarction.

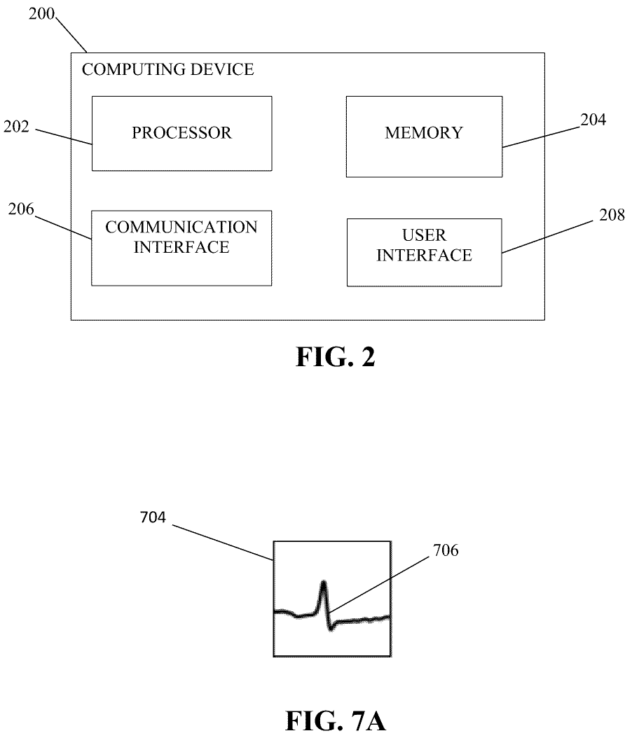

FIG. 2 illustrates a schematic of a computing device 200 including a processor 202 having code therein, a memory 204, and a communication interface 206. Optionally, the computing device 200 can include a user interface 208, such as an input device, an output device, or an input/output device. The processor 202, the memory 204, the communication interface 206, and the user interface 208 are operatively connected to each other via any known connections, such as a system bus, a network, etc. Any component, combination of components, and modules of the system 100 in FIG. 1 can be implemented by a respective computing device 200. For example, each of the hardware-based processor 102, the memory 104, the communication interface 106, the input/output device 108, and the set of modules 110-118 shown in FIG. 1 can be implemented by a respective computing device 200 shown in FIG. 2 and described below.

It is to be understood that the computing device 200 can include different components. Alternatively, the computing device 200 can include additional components. In another alternative implementation, some or all of the functions of a given component can instead be carried out by one or more different components. The computing device 200 can be implemented by a virtual computing device. Alternatively, the computing device 200 can be implemented by one or more computing resources in a cloud computing environment. Additionally, the computing device 200 can be implemented by a plurality of any known computing devices.

The processor 202 can be a hardware-based processor implementing a system, a sub-system, or a module. The processor 202 can include one or more general-purpose processors. Alternatively, the processor 202 can include one or more special-purpose processors. The processor 202 can be integrated in whole or in part with the memory 204, the communication interface 206, and the user interface 208. In another alternative implementation, the processor 202 can be implemented by any known hardware-based processing device such as a controller, an integrated circuit, a microchip, a central processing unit (CPU), a microprocessor, a system on a chip (SoC), a field-programmable gate array (FPGA), or an application-specific integrated circuit (ASIC). In addition, the processor 202 can include a plurality of processing elements configured to perform parallel processing. In a further alternative implementation, the processor 202 can include a plurality of nodes or artificial neurons configured as an artificial neural network. The processor 202 can be configured to implement any known machine learning (ML) based devices, any known artificial intelligence (AI) based devices, and any known artificial neural networks, including a recursive neural network (RNN) or a convolutional neural network (CNN).

The memory 204 can be implemented as a non-transitory computer-readable storage medium such as a hard drive, a solid-state drive, an erasable programmable read-only memory (EPROM), a universal serial bus (USB) storage device, a floppy disk, a compact disc read-only memory (CD-ROM) disk, a digital versatile disc (DVD), cloud-based storage, or any known non-volatile storage.

The code of the processor 202 can be stored in a memory internal to the processor 202. The code can be instructions implemented in hardware. Alternatively, the code can be instructions implemented in software. The instructions can be machine-language instructions executable by the processor 202 to cause the computing device 200 to perform the functions of the computing device 200 described herein. Alternatively, the instructions can include script instructions executable by a script interpreter configured to cause the processor 202 and computing device 200 to execute the instructions specified in the script instructions. In another alternative implementation, the instructions are executable by the processor 202 to cause the computing device 200 to execute an artificial neural network. The processor 202 can be implemented using hardware or software, such as the code. The processor 202 can implement a system, a sub-system, or a module, as described herein.

The memory 204 can store data in any known format, such as databases, data structures, data lakes, or network parameters of a neural network. The data can be stored in a table, a flat file, data in a filesystem, a heap file, a B+ tree, a hash table, or a hash bucket. The memory 204 can be implemented by any known memory, including random access memory (RAM), cache memory, register memory, or any other known memory device configured to store instructions or data for rapid access by the processor 202, including storage of instructions during execution.

The communication interface 206 can be any known device configured to perform the communication interface functions of the computing device 200 described herein. The communication interface 206 can implement wired communication between the computing device 200 and another entity. Alternatively, the communication interface 206 can implement wireless communication between the computing device 200 and another entity. The communication interface 206 can be implemented by an Ethernet, Wi-Fi, Bluetooth, or USB interface. The communication interface 206 can transmit and receive data over a network and to other devices using any known communication link or communication protocol.

The user interface 208 can be any known device configured to perform user input and output functions. The user interface 208 can be configured to receive an input from a user. Alternatively, the user interface 208 can be configured to output information to the user. The user interface 208 can be a computer monitor, a television, a loudspeaker, a computer speaker, or any other known device operatively connected to the computing device 200 and configured to output information to the user. A user input can be received through the user interface 208 implementing a keyboard, a mouse, or any other known device operatively connected to the computing device 200 to input information from the user. Alternatively, the user interface 208 can be implemented by any known touchscreen. The computing device 200 can include a server, a personal computer, a laptop, a smartphone, or a tablet.

Referring to FIG. 1, the vision transformer system 100 is configured to receive the ECG training data 122 at the communication interface 106 from the data source 120, and to store the received ECG training data 122 in the memory 104. The ECG training data 122 is processed by the processor 102 to train the transformer module 116 and optionally the MLP classification module 118. Once trained, the vision transformer system 100 receives, from the data source 120, the ECG data 124 corresponding to a patient at the communication interface 106. The vision transformer system 100 stores the received patient ECG data 124 in the memory 104. As described below, the trained vision transformer system 100 processes the patient ECG data 124 to generate and output, from the input/output device 108, a diagnosis 126 of the patient corresponding to the patient ECG data 124.

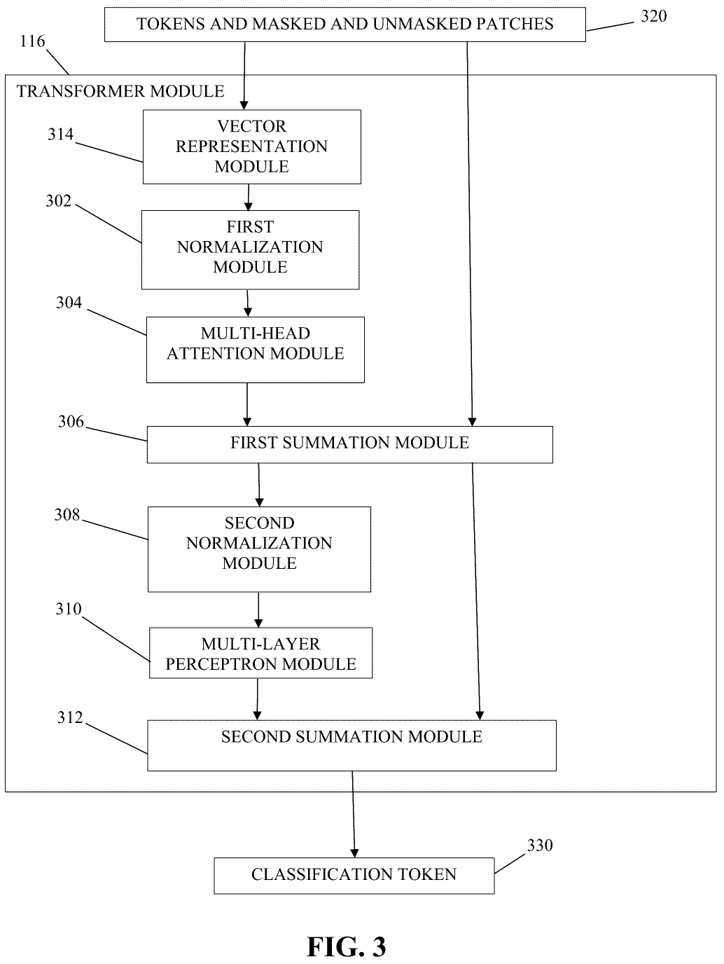

Referring to FIG. 3, the transformer module 116 shown in FIG. 1 includes a first normalization module 302, a multi-head attenuation module 304, a first summation module 306, a second normalization module 308, a multi-layer perceptron (MLP) module 310, and a second summation module 312. At least the multi-layer perceptron module 310 includes a first neural network configured to be responsive to received ECG training data 122 to be trained to generate a numerical classification token 330 from the tokens and the masked and unmasked patches 320, wherein the ECG training data 122 includes the plurality of training ECGs, with each training ECG including a plurality of pixels representing an image of at least one training ECG waveform.

As described below in connection with FIGS. 7-10, the patch generating module 110 generates a plurality of patches 700 from the ECGs in the ECG training data 122 or the patient ECG 124 by partitioning the ECG images of the ECG training data 112 or the patient ECG into a plurality of sub-images, wherein each patch is a respective sub-image having a fewer number of pixels than the ECG images.

The masking module 112 masks at least one of the plurality of patches 700 to generate a set of masked patches 904. The tokenization module 114 generates a plurality of tokens 800 from the plurality of patches 700 using a predetermined tokenization algorithm, with each numerical patch-based token 800 being a numerical value corresponding to a respective one of the plurality of patches 700. Referring back to FIG. 3, in one implementation, the transformer module 116 receives the tokens 800 and masked patches 904 to be the input tokens and masked and unmasked patches 320. In another implementation, the transformer module 116 receives and processes just the tokens 800. The set of masked patches 904 are used by the transformer module 116 at a later time to fine tune the transformer module 116. Optionally, the transformer module 116 includes a vector representation module 314 which converts the tokens 800 and positions of the tokens in the plurality of patches 700 into vector representations for processing by the transformer module 116.

In one implementation, the tokens and masked and unmasked patches 320, and optionally the corresponding vector representations, are input to the first normalization module 302 and the first summation module 306. The first normalization module 302 normalizes the tokens and masked and unmasked patches 320, and the multi-head attention module 304 receives the normalized tokens and masked and unmasked patches 320. Using a predetermined attention transformation, the multi-head attention module 304 concatenates and normalized tokens and masked and unmasked patches 320 and all of the attention outputs linearly to a predetermined set of dimensions. The many attention heads in the multi-head attention module 304 assist in training local and global dependencies in an image, such as the ECG 600. In one implementation, the predetermined attention transformation is an attention algorithm.

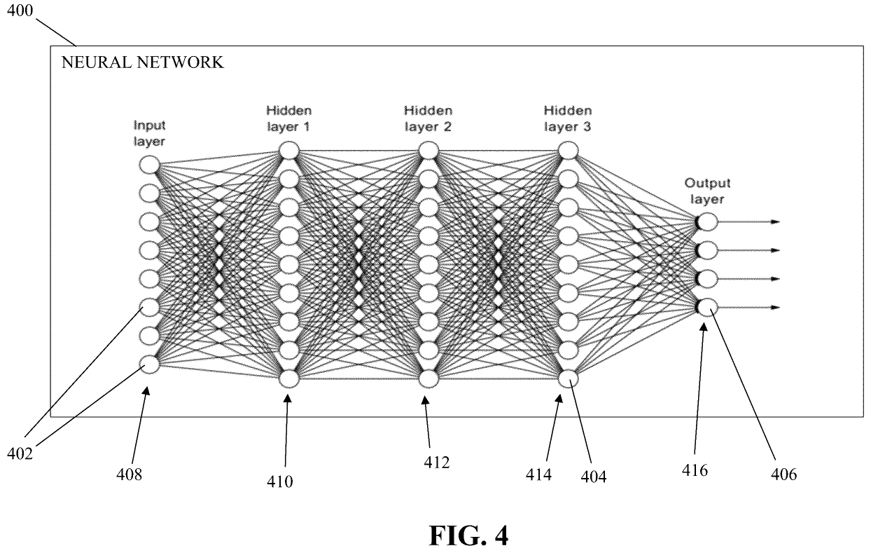

The concatenated and normalized tokens and masked patches are summed with the initial token and masked and unmasked patches 320, and optionally the corresponding vector representations, using the first summation module 306, and the summations are sent to the second normalization module 308 and the second summation module 312. The summations received by the second normalization module 308 are normalized, and the normalized summation is applied to the inputs of a multi-layer perceptron module 310. The multi-layer perceptron module 310 includes a feed-forward neural network such as the neural network 400 shown in FIG. 4. In one implementation consistent with the invention, the neural network 400 of FIG. 4 is a portion of an example architecture of the transformer module 116 and of the overall vision transformer system 100. In another implementation, other configurations of various known components including, but not limited to, neural networks are used in the transformer module 116.

Referring to FIG. 4, the neural network 400 includes a plurality of nodes or artificial neurons 402, 404, 406 arranged in a plurality of layers 408, 410, 412, 414, 416. The layer 408 is an input layer, and the layer 416 is an output layer, with the layers 410, 412, 414 being at least one hidden layer between input layer 408 and the output layer 416. In an implementation consistent with the invention, the neural network 400 of the transformer module 116 is a twelve layer transformer model with a hidden layer size of 768. In addition, in an implementation, the multi-head attention module 304 has twelve attention heads. Accordingly, with such a configuration of layers and attention heads, the transformer module 116 has a total of approximately 86 million parameters for performing as a vision transformer. In another implementation, the neural network 400 has other configurations of the nodes or artificial neurons 402, 404, 406 arranged in a different configuration of a plurality of layers.

The multi-layer perceptron module 410 processes the normalized summation to generate an output encoding which is output to the second summation module 312. In the second summation module 312, the output encoding is summed with the normalized summation from the first summation module 306 to generate a classification token 330. In an implementation, the transformer module 116 performs as a transformer using the components 302-312 to generate the classification token 330 from the tokens and masked and unmasked patches 320. In other implementations, the transformer module 116 implements a plurality of chains of the components 302-312 to carry out repeated transformations on the vector representations of the tokens and the positions of the tokens in the plurality of patches 800. Such repeated transformations extract more and more visual information concerning the ECGs. In one implementation, the plurality of chains of the components 302-312 include alternating attention layers and feedforward neural network layers.

After processing of the tokens and the masked and unmasked patches 320, the corresponding classification token 330 generated by the transformer module 116 is output to the multi-layer perceptron classification module 118. As with the multi-layer perceptron module 310 of the transformer module 116, the multi-layer perceptron classification module 118 includes a feed-forward neural network such as the neural network 400 shown in FIG. 4. Also, as with the multi-layer perceptron module 310 of the transformer module 116 trained by the ECG training data 122, the multi-layer perceptron classification module 118 is trained by the ECG training data 122. The trained multi-layer perceptron classification module 118 determines the diagnosis 126 from the classification token 330.

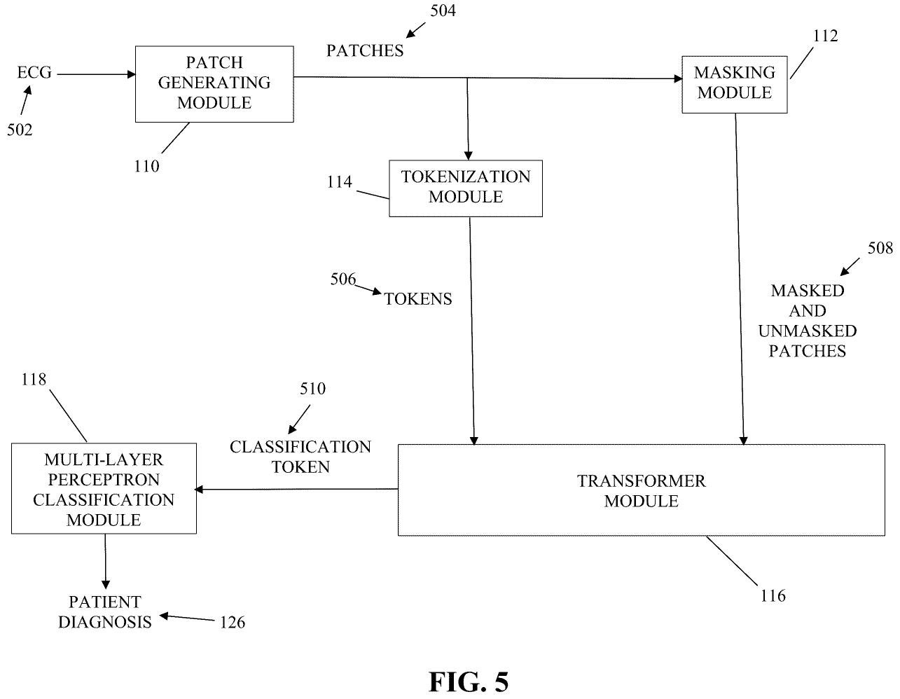

Referring to FIG. 5 in conjunction with FIGS. 3-4 and 6-10, the ECG 502, such as the ECG 600 in FIG. 6 having the waveforms 602, is input to the patch generating module 110 to generate a plurality of patches 504, such as the plurality of patches 700 in FIG. 7. As shown in FIG. 7, the patch generating module 110 partitions or otherwise generates the ECG 600 into a plurality of patches 700, with each patch of the plurality of patches 700 formed by overlaying a grid having grid lines 702 onto the image data of the ECG 600. As shown in FIG. 7A, an example patch 704 is a sub-image derived from the partitioning of the original ECG 600. In one implementation, for the patch 704, a portion of the ECG waveform 706 is present. In another implementation, the patches lack any waveform, such as the patches 708 shown in a top row of FIG. 7.

In an implementation consistent with the invention, the patch generating module 110 partitions the image of the ECG 600 into M×N sub-images, with M and N specifying the row dimension and column dimension, respectively, of each sub-image as a patch. In one implementation, M and N are integers. For example, with M and N equal to 14, 196 sub-images are generated. In one implementation, the plurality of patches 700 are arranged in a grid of 196 squares, forming a 14×14 grid of patches, with each patch having 16×16 pixels of the original ECG 600. In one implementation, the values of M and N are set by default patch dimensions to be, for example, 14. In another implementation, a system administrator, using the input/output device 108, sets or changes the values of the patch dimensions M and N. Accordingly, different values of M and N are adjustable, allowing for greater granularity in the processing of the ECG 600 and its waveforms 602.

Referring back to FIG. 5, the patches 504 are output from the patch generating module 110 to be received by the masking module 112 and the tokenization module 114 to generate masked and unmasked patches 508 and tokens 506, respectively, such as the masked patch 904 and the tokens 800, in FIGS. 9 and 8, respectively. As shown in FIG. 8, each of the tokens 800 is a set of data values representing or encoding a respective one of the plurality of patches 700 in FIG. 7. Accordingly, in one implementation, the tokenization module 114 generates 196 tokens corresponding to the 196 patches of the plurality of patches 700, respectively, in the 14×14 grid of patches. In the implementation, the tokens 800 in FIG. 8 are arranged in a matrix format, such as a 14×14 matrix, with the matrix of tokens 800 stored in the memory 104. In another implementation, the tokens 800 in FIG. 8 are arranged in any known data format, such as 14 sets of 14×1 vectors stored in the memory 104.

In an implementation consistent with the invention, the tokenization module 114 generates the tokens 800 using the DALL-E generative pre-trained transformer system publicly available from OPENAI, INC., such that DALL-E generates each of the tokens 800 from a respective patch of the plurality of patches 700. In another implementation, the tokenization module 114 generates each of the tokens 800 from a respective patch of the plurality of patches 700 using any known image-to-data generating technique or tokenization algorithm for tokenizing the plurality of patches 700 derived from the image of the ECG 600.

Referring to FIGS. 5 and 9-10, the masking module 112 generates the masked and unmasked patches 508 from the patches 504, which is represented in FIG. 9 as a 14×14 grid of patches 900 having unmasked patches 902 and masked patches 904. Each masked patch 904 includes a plurality of pixels having a predetermined color, such as black as shown in FIGS. 9-10. In one implementation, the color black is a default masking color. In another implementation, a system administrator, using the input/output device 108, sets or changes the default value or setting of the masking color to be used by the masking module 112. As shown in FIG. 9, the masked patches 904 replace the pixels of the image in a given patch with a patch of black pixels. For example, a preset percentage or portion of the number of tokens input to the transformer module 116 are masked or hidden, and the transformer module 116 is pre-trained by having the transformer module 116 predict such masked tokens. In an implementation consistent with the invention, the preset percentage of masking tokens is set at a default value of 40% of the input patches 700 which are masked for input into the neural network of the transformer module 116. In another implementation, a system administrator, using the input/output device 108, sets or changes the default value of the preset percentage or portion to be used by the masking module 112. In one implementation, an Adaptive Moment Estimation (Adam) optimization technique is used to control the amount of masking. For example, the AdamW optimizer is used as the optimization technique which is a stochastic optimization method that modifies the typical implementation of weight decay, and using a learning rate of 5×10⁻⁴. In an implementation, the masking is performed to cut out attention links between some image pairs.

As shown in FIGS. 9-10, an example, segment 906 of the patches 900 includes a plurality of patches 1000 shown in FIG. 10, with some patches 1002 remaining unmasked, while other patches 1004 are masked. Referring to FIGS. 8 and 10, the tokens 800 include tokens 802 which are not highlighted and which represent unmasked patches such as the unmasked patch 1002. Other tokens 804 are highlighted to represent masked patches such as the masked patch 1004. Accordingly, referring to FIGS. 5 and 8-10, each of the tokens 800 corresponds to masked or unmasked patches 900, such that each of the tokens 506 corresponds to one of the masked and unmasked patches 508.

Referring back to FIGS. 3 and 5, the transformer module 116 receives the tokens 506 and the masked and unmasked patches 508, as the tokens and the masked and unmasked patches 320 in FIG. 3, to generate the classification token 510, corresponding to the classification token 330 in FIG. 3. The classification token 510 is applied to the multi-layer perceptron classification module 118 to generate the patient diagnosis 126. The multi-layer perceptron classification module 118 includes a second trained neural network configured to be responsive to received ECG training data 122 to be trained to generate and output a diagnosis message from the numerical classification token 330, 510. The multi-layer perceptron classification module 118 is responsive to the patient ECG 124 to generate the diagnosis message as a patient diagnosis corresponding to the patient ECG 124 and indicating a state of health of the heart of the patient. In one implementation, the patient diagnosis 126 is a numerical value which is compared to a cut-off value as described above. In another implementation, the patient diagnosis 126 is a text message or a message in other media such as an audio message, a video message, an animation, a chart, etc. indicating the state of health of the heart of the patient. For example, the patient diagnosis 126 is a text message or a message in other media generated from a numerical value determined by the multi-layer perceptron classification module 118 using a predetermined algorithm.

In another implementation consistent with the invention, the vision transformer system 100 includes additional and known components, systems, and applications are used to convert the patient diagnosis 126, in numerical form, to a text message or a message in other media. For example, the vision transformer system 100 includes a known natural language processing (NLP) system to convert the patient diagnosis 126 in numerical form to a text message or a message in other media for output by the vision transformer system 100. In another example, the vision transformer system 100 includes a known generative pre-trained transformer (GPT) system, such as the DALL-E system, to convert the patient diagnosis 126 in numerical form to a text message or a message in other media for output by the vision transformer system 100. In an implementation, the NLP system or the GPT system generate the text message or the message in other media as the diagnosis message in the patient diagnosis 126, with such a diagnosis message indicating a healthy heart, an unhealthy heart, or a specific condition such as hypertrophic cardiomyopathy, low left ventricular ejection fraction, or ST elevation myocardial infarction. The generated the text message or the message in other media as the diagnosis message is then output by the vision transformer system 100.

During training of the transformer module 116 and the multi-layer perceptron classification module 118, the ECG training data 122 is applied to the vision transformer system 100 to generate initial patent diagnoses 126 for training purposes. In one implementation, the transformer module 116 and the multi-layer perceptron classification module 118 are trained using back propagation techniques. In an implementation, the training of the transformer module 116 and the multi-layer perceptron classification module 118 is performed by iteratively or repeatedly applying the ECG training data 122 to vision transformer system 100, and comparing the resulting initial patient diagnoses 126 during training to actual patient diagnoses in the ECG training data 122 until the initial patient diagnoses 126 as training generated diagnoses differ from an actual training diagnosis in the ECG training data 122 to be within a predetermined training threshold. In one implementation, the predetermined training threshold is a default value of 5%. The training generated diagnosis has a numerical training value, and the actual training diagnosis has a numerical actual value. Accordingly, when the numerical training value is within 5% of the numerical actual value, the transformer module 116 or the multi-layer perceptron classification module 118 are trained. In one implementation, the predetermined training threshold is set to a default value. In another implementation, a system administrator, using the input/output device 108, sets or changes the value of the predetermined training threshold.

In another implementation, the transformer module 116 and the multi-layer perceptron classification module 118 are trained using gradient descent techniques. In a further implementation, the transformer module 116 and the multi-layer perceptron classification module 118 are trained using any known machine learning and training technique to train the transformer module 116 and the multi-layer perceptron classification module 118, and thus the vision transformer system 100 to predict or infer the patient diagnosis 126 from the patient ECG 124.

In addition, the transformer module 116 and in turn the vision transformer system 100 are trained using self-supervised learning involving unsupervised pre-training followed by supervised fine-tuning. In an implementation, the transformer module 116 and in turn the vision transformer system 100 undergo transfer learning such that the transformer module 116 and in turn the vision transformer system 100 are trained on a larger, possibly unrelated dataset and then fine-tuned on a smaller dataset that is relevant to a problem, such as diagnosing a patient and the health of the heart of the patient from the patient ECG 124. Transfer learning is especially useful in healthcare since datasets are limited in size due to limited patient cohorts, rarity of outcomes of interest, and costs associated with generating useful labels.

In one implementation, the Adam optimizer on a OneCycle learning rate schedule between 3×10⁻⁴ and 1×10⁻³ over thirty epochs is utilized for fine-tuning the vision transformer system 100 and for reporting performance metrics corresponding to the best performance achieved across the thirty epochs. In an implementation consistent with the invention, analyses of data and performance of the vision transformer system 100 use pandas, numpy, Python Image Library (PIL), SciPy, scikit-learn, torchvision, timm, and PyTorch libraries.

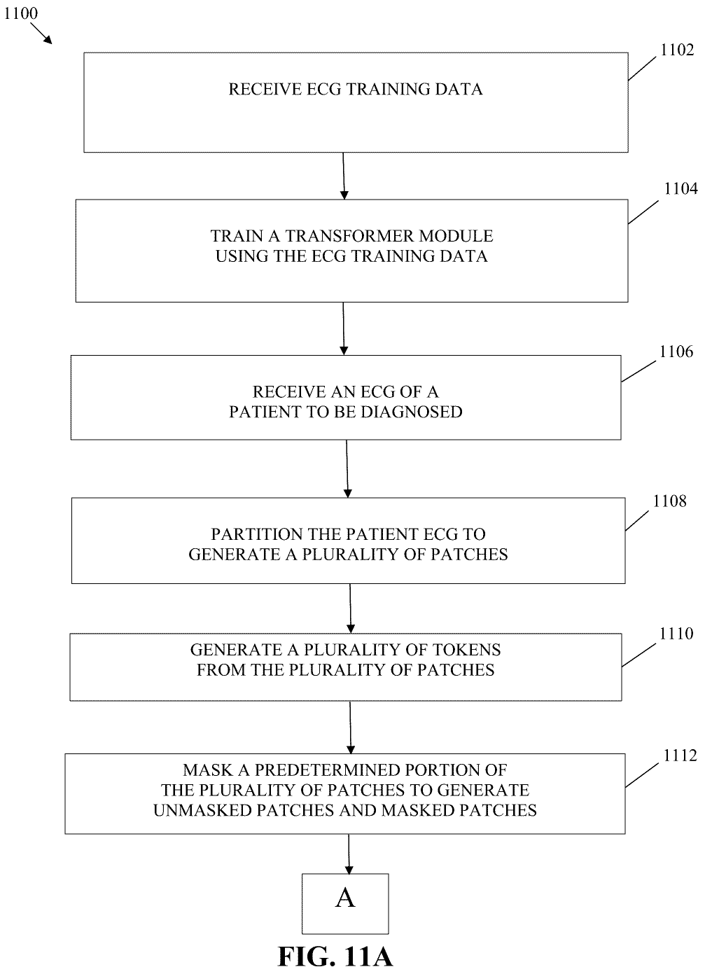

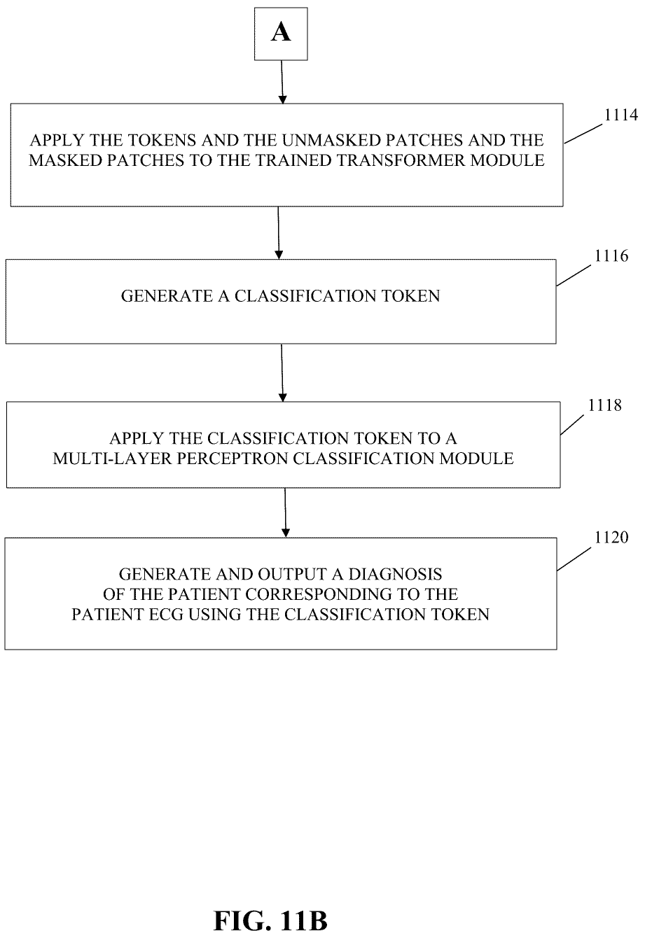

Referring to FIGS. 11A-11B, in an implementation consistent with the invention, a computer-based method 1100 implements the vision transformer system 100 configured to generate a patient diagnosis 126 from the patient ECG 124, with the patient ECG 124 includes a plurality of pixels representing an image having at least one patient ECG waveform. The computer-based method 1110 includes receiving the ECG training data 122 in step 1102, training at least the transformer module 116 using the ECG training data 122 in step 1104, and receiving an ECG 124 of a patient to be diagnosed, such as the ECG 600 in step 1106. The computer-based method 1100 then partitions the patient ECG 124 in step 1108 to generate a plurality of patches 700 shown in FIG. 7. The computer-based method 1100 then generates a plurality of tokens 800 from the plurality of patches 700 in step 1110. The computer-based method 1100 then masks a predetermined portion of the plurality of patches 700 in step 1112 to generate a plurality of patches 900 including unmasked patches 902 and masked patches 904 as shown in FIG. 9.

The computer-based method 1100 then applies the plurality of tokens 800 and the plurality of patches 900 including the unmasked patches 902 and the masked patches to the trained transformer module 116 in step 1114, and the trained transformer module 116 generates a classification token 330, 510 in step 1116. The computer-based method 1100 then applies the classification token 330, 510 to the multi-layer perceptron classification module 118 in step 1118, and the multi-layer perceptron classification module 118 generates and outputs a diagnosis of the patient corresponding to the patient ECG 124 using the classification token 330, 510 in step 1120. Using the input/output device 108, the generated and output diagnosis is displayed on a display or monitor, or otherwise output by the vision transformer system 100, as described above.

In an implementation consistent with the invention, a non-transitory computer-readable storage medium stores instructions executable by a processor to implement the vision transformer system 100 and method 1100 configured to generate a patient diagnosis 126 from the patient ECG 124, with the patient ECG 124 includes a plurality of pixels representing an image having at least one patient ECG waveform. The instructions include receiving the ECG training data 122, training at least the transformer module 116 using the ECG training data 122, receiving an ECG 124 of a patient to be diagnosed such as the ECG 600, partitioning the patient ECG 124 to generate a plurality of patches 700 shown in FIG. 7, generating a plurality of tokens 800 from the plurality of patches 700, masking a predetermined portion of the plurality of patches 700 to generate a plurality of patches 900 including unmasked patches 902 and masked patches 904 as shown in FIG. 9, applying the plurality of tokens 800 and the plurality of patches 900 including the unmasked patches 902 and the masked patches to the trained transformer module 116, generating classification token 330, 510 using the trained transformer module 116, applying the classification token 330, 510 to the multi-layer perceptron classification module 118, and generating and outputting, from the multi-layer perceptron classification module 118, a diagnosis 126 of the patient corresponding to the patient ECG 124 using the classification token 330, 510. In another implementation, the instructions including outputting the patient diagnosis 126 on a display or monitor, or otherwise output by the vision transformer system 100, as described above.

Portions of the methods described herein can be performed by software or firmware in machine readable form on a tangible or non-transitory storage medium. For example, the software or firmware can be in the form of a computer program including computer program code adapted to cause the system to perform various actions described herein when the program is run on a computer or suitable hardware device, and where the computer program can be implemented on a computer readable medium. Examples of tangible storage media include computer storage devices having computer-readable media such as disks, thumb drives, flash memory, and the like, and do not include propagated signals. Propagated signals can be present in a tangible storage media. The software can be suitable for execution on a parallel processor or a serial processor such that various actions described herein can be carried out in any suitable order, or simultaneously.

It is to be further understood that like or similar numerals in the drawings represent like or similar elements through the several figures, and that not all components or steps described and illustrated with reference to the figures are required for all embodiments, implementations, or arrangements.

The terminology used herein is for the purpose of describing particular implementations only and is not intended to be limiting of the invention. As used herein, the singular forms “a,” “an,” and “the” are intended to include the plural forms as well, unless the context clearly indicates otherwise. It will be further understood that the terms “contains”, “containing”, “includes”, “including,” “comprises”, and/or “comprising,” and variations thereof, when used in this specification, specify the presence of stated features, integers, steps, operations, elements, and/or components, but do not preclude the presence or addition of one or more other features, integers, steps, operations, elements, components, and/or groups thereof.

Terms of orientation are used herein merely for purposes of convention and referencing and are not to be construed as limiting. However, it is recognized these terms could be used with reference to an operator or user. Accordingly, no limitations are implied or to be inferred. In addition, the use of ordinal numbers (e.g., first, second, third) is for distinction and not counting. For example, the use of “third” does not imply there is a corresponding “first” or “second.” Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

While the disclosure has described several exemplary implementations, it will be understood by those skilled in the art that various changes can be made, and equivalents can be substituted for elements thereof, without departing from the spirit and scope of the invention. In addition, many modifications will be appreciated by those skilled in the art to adapt a particular instrument, situation, or material to implementations of the disclosure without departing from the essential scope thereof. Therefore, it is intended that the invention not be limited to the particular implementations disclosed, or to the best mode contemplated for carrying out this invention, but that the invention will include all implementations falling within the scope of the appended claims.

The subject matter described above is provided by way of illustration only and should not be construed as limiting. Various modifications and changes can be made to the subject matter described herein without following the example embodiments, implementations, and applications illustrated and described, and without departing from the true spirit and scope of the invention encompassed by the present disclosure, which is defined by the set of recitations in the following claims and by structures and functions or steps which are equivalent to these recitations.

What is claimed is:

1. A vision transformer system configured to generate a patient diagnosis, the vision transformer system comprising:
a hardware-based processor;
a memory configured to store instructions and configured to provide the instructions to the hardware-based processor; and
a set of modules configured to implement the instructions provided to the hardware-based processor, the set of modules including:
a patch generating module configured to generate a plurality of patches by partitioning an image of a patient electrocardiogram (ECG) having at least one patient ECG waveform into a plurality of sub-images, wherein each patch is a respective one of the plurality of sub-images and further wherein each patch has fewer pixels than the image of the patient ECG;
a tokenization module configured to generate, using a predetermined tokenization algorithm, a plurality of numerical patch-based tokens, wherein each of the numerical patch-based tokens is a numerical value representing a respective one of the plurality of patches;
a transformer module configured to generate, by processing the plurality of numerical patch-based tokens, a numerical classification token representing the patient ECG; and
a classification module configured to generate and output, by processing the numerical classification token, a diagnostic message representing a patient diagnosis corresponding to the patient ECG and indicating a state of health of the heart of the patient.

2. The vision transformer system of claim 1, wherein the transformer module includes a first neural network that is trained using ECG training data including an image of at least one training ECG having at least one training ECG waveform, and further wherein the first neural network is trained by repeatedly evaluating the ECG training data until a respective first training generated diagnosis differs from an actual first training diagnosis within a first predetermined training threshold.

3. The vision transformer system of claim 2, wherein the classification module includes a second neural network that is trained using the ECG training data, and further wherein the second neural network is trained by repeatedly evaluating the ECG training data until a respective second training generated diagnosis differs from an actual second training diagnosis within a second predetermined training threshold.

4. The vision transformer system of claim 3, wherein the classification module comprises a multi-layer perceptron classification module including the second trained neural network.

5. The vision transformer system of claim 3, wherein the transformer module comprises:
a multi-head attention module configured to perform a predetermined attention transformation on the plurality of numerical patch-based tokens; and
a multi-layer perceptron module including the first trained neural network configured to generate the numerical classification token from the transformed plurality of numerical patch-based tokens.

6. The vision transformer system of claim 1, further comprising:
a masking module configured to generate, by masking a subset of the plurality of patches, a plurality of masked patches, wherein the transformer module is configured to generate the numerical classification token using:
the plurality of numerical patch-based tokens;
a plurality of unmasked patches; and
the plurality of masked patches.

7. The vision transformer system of claim 6, wherein each of the masked patches includes pixels having a predetermined color.

8. The vision transformer system of claim 6, wherein the masking module includes an optimizer configured to perform stochastic optimization with a predetermined learning rate to define the subset of the plurality of patches.

9. The vision transformer system of claim 1, wherein the tokenization module includes a generative pre-trained transformer configured to convert each of the plurality of patches to respective ones of the plurality of numerical patch-based tokens.

10. A pre-trained vision transformer system configured to generate a patient diagnosis, the vision transformer system comprising:
a hardware-based processor;
a memory configured to store instructions and configured to provide the instructions to the hardware-based processor; and
a set of modules configured to implement the instructions provided to the hardware-based processor, the set of modules including:
a patch generating module configured to generate a plurality of patches by partitioning an image of a patient electrocardiogram (ECG) having at least one patient ECG waveform into a plurality of sub-images, wherein each patch is a respective one of the plurality of sub-images and further wherein each patch has fewer pixels than the image of the patient ECG;
a tokenization module configured to generate, using a predetermined tokenization algorithm, a plurality of numerical patch-based tokens, wherein each of the numerical patch-based tokens is a numerical value representing a respective one of the plurality of patches;
a pre-trained transformer module trained using ECG training data including an image of at least one training ECG with at least one training ECG waveform, and configured to generate, by processing the plurality of numerical patch-based tokens, a numerical classification token representing the patient ECG; and
a pre-trained classification module trained using the ECG training data and configured to generate and output, by processing the numerical classification token, a diagnostic message representing a patient diagnosis corresponding to the patient ECG and indicating a state of health of the heart of the patient.

11. The pre-trained vision transformer system of claim 10, wherein the pre-trained transformer module includes a first neural network that is trained using the ECG training data, and further wherein the first neural network is trained by repeatedly evaluating the ECG training data until a respective first training generated diagnosis differs from an actual first training diagnosis within a first predetermined training threshold.

12. The pre-trained vision transformer system of claim 11, wherein the pre-trained classification module includes a second neural network that is trained using the ECG training data, and further wherein the second neural network is trained by repeatedly evaluating the ECG training data until a respective second training generated diagnosis differs from an actual second training diagnosis within a second predetermined training threshold.

13. The pre-trained vision transformer system of claim 12, wherein the classification module comprises a multi-layer perceptron classification module including the second trained neural network.

14. The pre-trained vision transformer system of claim 12, wherein the transformer module comprises:
a multi-head attention module configured to perform a predetermined attention transformation on the plurality of numerical patch-based tokens; and
a multi-layer perceptron module including the first trained neural network configured to generate the numerical classification token from the transformed plurality of numerical patch-based tokens.

15. The pre-trained vision transformer system of claim 10, further comprising:
a masking module configured to generate, by masking a subset of the plurality of patches, a plurality of masked patches, wherein the transformer module is configured to generate the numerical classification token using:
the plurality of numerical patch-based tokens;
a plurality of unmasked patches; and
the plurality of masked patches.

16. The pre-trained vision transformer system of claim 15, wherein each of the masked patches includes pixels having a predetermined color.

17. The pre-trained vision transformer system of claim 15, wherein the masking module includes an optimizer configured to perform stochastic optimization with a predetermined learning rate to define the subset of the plurality of patches.

18. The pre-trained vision transformer system of claim 10, wherein the tokenization module includes a generative pre-trained transformer configured to convert each of the plurality of patches to respective ones of the plurality of numerical patch-based tokens.

19. A computer-based method, comprising:
receiving an electrocardiogram (ECG) of the patient, wherein the patient ECG includes a plurality of pixels representing an image having at least one patient ECG waveform;
generating a plurality of patches of the patient ECG by partitioning the image of the patient ECG into a plurality of sub-images, wherein each patch is a respective one of the plurality of sub-images and further wherein each patch has fewer pixels than the image of the patient ECG;
generating a plurality of numerical patch-based tokens from the plurality of patches using a predetermined tokenization algorithm, wherein each numerical patch-based token is a numerical value representing a respective one of the plurality of patches;
generating a numerical classification token by processing the plurality of numerical patch-based tokens using a transformer module having a first trained neural network, wherein the numerical classification token represents the patient ECG;
generating a diagnosis message from the numerical classification token processed by a classification module including a second trained neural network, wherein the diagnosis message represents a patient diagnosis corresponding to the patient ECG and indicating a state of health of the heart of the patient; and
outputting the diagnosis message.

20. The computer-based method, comprising:
providing a first neural network in the transformer module;
providing a second neural network in the classification module;
training the first neural network using ECG training data including an image of at least one training ECG having at least one training ECG waveform, wherein the training of the first neural network includes:
repeatedly evaluating the ECG training data until a respective first training generated diagnosis differs from an actual first training diagnosis within a first predetermined training threshold; and
training the second neural network using the ECG training data, wherein the training of the second neural network includes:
repeatedly evaluating the ECG training data until a respective second training generated diagnosis differs from an actual second training diagnosis within a second predetermined training threshold.
